(19) United States
(12) Patent Application Publication
(10) Pub. No.: US 2004/0006329 A1
(43) Pub. Date: Jan. 8, 2004

(54) Device for holding and guiding a guide wire in a catheter

(76) Inventor: Rolf Rainer Scheu, Frankfurt (DE)

Correspondence Address: Pauley Petersen Kinne & Erickson, Suite 365, 2800 W. Higgins Road, Hoffman Estates, IL, 60195, US

(21) Appl. No.: 10/281,294

(22) Filed: Oct. 25, 2002

(30) Foreign Application Priority Data
Jul. 5, 2002 (DE) 202 10 509.1
Sep. 28, 2002 (DE) 202 15 003.8

(51) Int. Cl. A61M 25/01

(52) U.S. Cl. 604/528

(57) ABSTRACT

A device for holding and guiding a guide wire in a catheter. The device can be secured releasably to the guide wire, having a proximal end and a distal end, and having a recess that is continuous from the proximal end to the distal end of the device. The guide wire passes through the recess in order to introduce the catheter into a vessel or a body cavity of a patient. The device releasably secures the catheter, so that a common motion of the guide wire and catheter, in a previously defined position of the guide wire relative to the catheter, can be performed by the device.

### BACKGROUND OF THE INVENTION

1. Field of the Invention

This invention relates to a device for holding and guiding a guide wire in a catheter, which device can be secured releasably to the guide wire, having a proximal end and a distal end, and having a recess, and that is continuous from the proximal end to the distal end of the device, for passing the guide wire through in order to introduce the catheter into vessels or body cavities of a patient.

2. Discussion of Related Art

In medical technology, guide wires in catheters are used in manifold ways, such as to introduce catheters into vessels of a patient. It is known to provide the guide wire, on its proximal end that protrudes out of the catheter, with a device known as torque control for fixation and holding, as shown in FIG. 2. The guide wire 1 provided on its proximal end with the holder in the form of the rotary grip G, is passed through the continuous lumen 24 in the catheter 2 up to the distal end 22 of the catheter and beyond and is used, for example in cardiological angiography to find vessel branches. The guide wire 1 is first thrust into the blood vessel 30, and then the catheter 2 is slipped over the guide wire 1 into the blood vessel 30. For finding a vessel branch 32 and introduce the catheter into it, the guide wire 1 can now, by grasping the rotary handle G, be rotated about its axis in the rotational direction of the arrow D, and as a result the tip I a, here bent at an angle, for instance, of the guide wire also rotates and can thus be introduced into the branch 32.

One disadvantage of a known torque control device, for holding and guiding the guide wire for placement of a catheter or a probe is that because the guide wire can shift axially with respect to the catheter. Thus an intended position of the guide wire can vary during a treatment, and a guide wire displaced axially in the catheter can emerge through lateral eyelets on the catheter, or guide wires without a bent tip or that emerge too far from the catheter on the distal end can lead to a risk of injury to the patient.

### SUMMARY OF THE INVENTION

It is one object of this invention to provide a device for guiding and holding a guide wire for introducing and positioning a catheter, so that undesired axial shifting of the guide wire and catheter relative to one another is not possible, thus also lessening the risk of injury.

According to the invention, this object is attained with a device as described in this specification and in the claims.

In this specification and in the claims, the term probes are also understood to be covered by the term catheters.

According to this invention, a device for holding and guiding a guide wire in a catheter is proposed, which can releasably secure the catheter, so that with the device a common motion of the guide wire and catheter, in a previously defined position of the guide wire relative to the catheter, can be performed.

According to this invention, a catheter and guide wire fixation not only holds a guide wire but also simultaneously makes it possible to secure the catheter to the holder. Such an arrangement allows a common simultaneous movement of the guide wire and the catheter, or in other words a common change in position, with a previously defined position of the guide wire relative to the catheter, and maintenance of this position, even during common motion, so that undesired axial shifting of the guide wire relative to the catheter can no longer occur. In this way, a novel and substantially improved and secure placement technique for placing catheters and probes with a guide wire is achieved.

The catheter and guide wire fixation of this invention thus not only hold the guide wire but also simultaneously hold the catheter or probe. The common motion has an advantage in terms of manipulating the device. At the same time, the position, once defined, of the guide wire and thus of the tip of the guide wire relative to the associated catheter does not vary. This avoids risks of injury from the guide wire. In the known device shown in FIG. 2, a guide wire displaced in the catheter can sometimes emerge through lateral eyelets of a catheter, or through the catheter if the catheter has an open tip.

According to this invention, there is a fixation device for passing through and fixing the guide wire. The fixation device includes a housing, a squeezer element that can be inserted into the housing. A closure part can be connected releasably to the housing. The means for releasably securing the catheter are disposed on the distal end of the fixation device.

The means for releasably securing the catheter can be a catheter adapter with a catheter connector for connecting the catheter that is secured releasably to the distal end of the fixation device.

In a further embodiment of this invention, a catheter connector for connecting the catheter is embodied integrally with the fixation device. The catheter connector can be on the distal end of the fixation device.

The catheter adapter with a catheter connector for the releasable securing to the fixation device, or the forming on or embodiment of a catheter connector directly on the fixation device, can be embodied in manifold forms and other known forms. All the usual catheter connectors may be contemplated, such as the catheter connector being a flexible-tube coupling, or a male or female Luer-lock connector, or a stepped connector.

The fixation device for the guide wire, according to this invention, includes a housing, embodied as a grip, with a continuous bore for the passage of the guide wire, a squeezer element with a continuous bore for the passage of the guide wire, and a closure part, embodied as a punch for action on the squeezer element, with a continuous bore. The squeezer element is insertable into the bore of the housing, and the closure part can be secured releasably to the housing by exerting a pressure on the squeezer element. The bores of the housing and of the squeezer element and of the closure part extend in a common axis.

The distal end of the fixation device is formed by the closure part. In accordance with one embodiment of this invention, for securing and holding the catheter on the fixation device there is a catheter adapter with a catheter connector, which can be secured to the distal end of the fixation device, particularly to the distal end of the closure part. Thus, the distal end of the closure part can be embodied as a connecting piece for the connection of a catheter adapter, for example as a Luer-lock connecting piece.

For a releasable embodiment, the catheter connector is formed onto the catheter adapter.

In another embodiment of this invention, instead of a separate catheter adapter for holding the catheter, the distal end of the fixation device, in this case the distal end of the closure part itself, is embodied as a catheter connector. The catheter connector is formed directly onto the distal end of the closure part, and then forms a unit with the closure part. The connecting piece of the catheter to the closure part embodied integrally with a catheter connector can then be accomplished, for instance via a flexible-tube coupling embodied on the closure part, or via a Luer-lock connector, or via an embodiment as a stepped connector.

The closure part, on whose distal end the catheter adapter is secured releasably, or whose distal end itself is embodied as a catheter connector, is connected releasably on its proximal end to the housing using a screw connection, a bayonet mount, and/or a detent and/or clamping connection.

The housing is preferably embodied cylindrically or spherically and is embodied on the outside, to improve the grip with profiling, such as axially extending, protruding ribs distributed uniformly or the circumference, or fluting. For connecting the housing to the closure part, the closure part in its distal end region, for example, can have a collar that with respect to the housing is offset and has a reduced diameter; on the outside, the collar has profiling, such as a thread or as grooves, beads, and/or channels for forming a positive engagement with the closure part.

According to this invention, the closure part can be embodied with a nozzle stub with a continuous bore, the nozzle stub being continuous from the proximal end to the distal end of the closure part. The proximal end region of the nozzle stub is embodied for introduction into the bore of the housing. The end face, embodied on the proximal end, of the nozzle stub acts as a contact-pressure face upon the squeezer element. The proximal end of the nozzle stub is surrounded with clearance by a coaxially disposed jacket, forming an annular chamber. A thread or profiling for releasable connection to the housing is embodied on the inside of the jacket. For connecting the closure part to the housing while exerting a squeezing and crushing pressure on the squeezer element, the collar of the housing is embodied so that it can be introduced into the annular chamber formed on the closure part between the jacket and the proximal nozzle stub.

For secure seating in the housing and for exerting a pressure on the squeezer element, the continuous bore in the housing is embodied as tapering in stepped fashion, beginning at least at the distal end of the housing, toward the middle of the housing, and on the steplike shoulder a bearing face for the squeezer element is formed.

The squeezer element can be made from an elastic material, such as a thermoplastic and/or elastomeric plastic, silicone, rubber, or a natural rubber, with a continuous bore, so that when a suitable pressure is exerted, crushing can occur, as a result of which the guide wire, passed through the bore, as firmly clamped by a reduction in the bore diameter.

However, it is also possible to make the squeezer element from a hard material such as hard plastic or metal, which is not compressible but with suitable shaping, such as slots, is embodied as resiliently compressible, as a result of which, once again, by a reduction in the diameter of a continuous bore, a guide wire can be firmly clamped therein.

In another embodiment of this invention, a catheter adapter with a catheter connector is releasably secured to the distal end of the fixation device, or a catheter connector is formed onto the distal end of the fixation device and a catheter is connected to the catheter connector. The position of the catheter relative to the fixation device is defined, and the guide wire guided through the fixation device is squeezed in the squeezer element by securing the closure part to the housing and is fixed in its position relative to the fixation device and to the catheter secured to it. A simultaneous common change of position of the catheter and guide wire can be performed while maintaining the previously defined position of the guide wire relative to the catheter.

It is thus possible for the fixation device of this invention, for the guide wire and the catheter adapter to be used for simultaneously firmly holding a guide wire and the catheter and performing a common forward and backward motion in the axial direction while preventing the guide wire and catheter from shifting counter to one another in the axial direction.

This invention is advantageous, for example, for gastral and jejunal probes with an open tip or an open distal end. Gastral and jejunal probes are also produced with a closed tip, so that the guide wire on being advanced cannot unintentionally emerge beyond the distal end of the probe. A probe tip or catheter tip that is closed at the distal end, however, has one disadvantage that administered medications can sometimes be trapped at the probe tip, because they can leave only through the lateral eyelets in the probe, which can make the medical therapy incorrect, for instance.

### BRIEF DESCRIPTION OF THE DRAWINGS

This invention is described in further detail below in conjunction with the drawing, in terms of an exemplary embodiment, wherein:

FIG. 1a shows a fixation device for a guide wire and a catheter, in longitudinal section, in a closed position with a separate catheter adapter and catheter connector;

FIG. 1b shows a fixation device for a guide wire and a catheter, in longitudinal section, in a closed position with an integrated catheter adapter;

FIG. 2 shows a conventional device for holding and guiding a guide wire, in a schematic view;

FIG. 3 shows the fixation device for the guide wire and the catheter of this invention, in an exploded view in longitudinal section as in FIG. 1a, but in an opened position with a catheter adapter;

FIG. 4 shows the fixation device for holding the guide wire of FIG. 3, but in a closed position without a catheter adapter;

FIGS. 5a-5c and 6a-6c show a sectional view, a plan view and a front view, respectively, each of a different exemplary embodiment of the squeezer element;

FIG. 7 shows the housing of FIG. 4 on a smaller scale, in a plan view; and

FIGS. 8 and 9 each shows a sectional view of a different embodiment of a catheter adapter for connection to the fixation device of FIG. 4.

### DESCRIPTION OF PREFERRED EMBODIMENTS

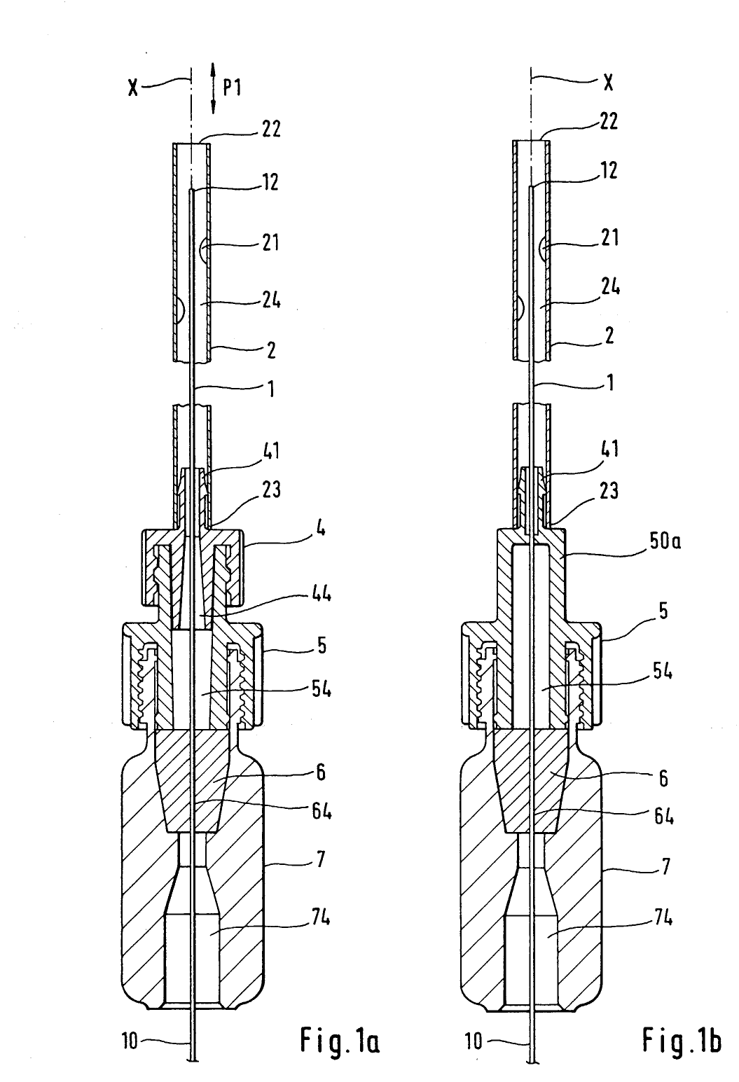

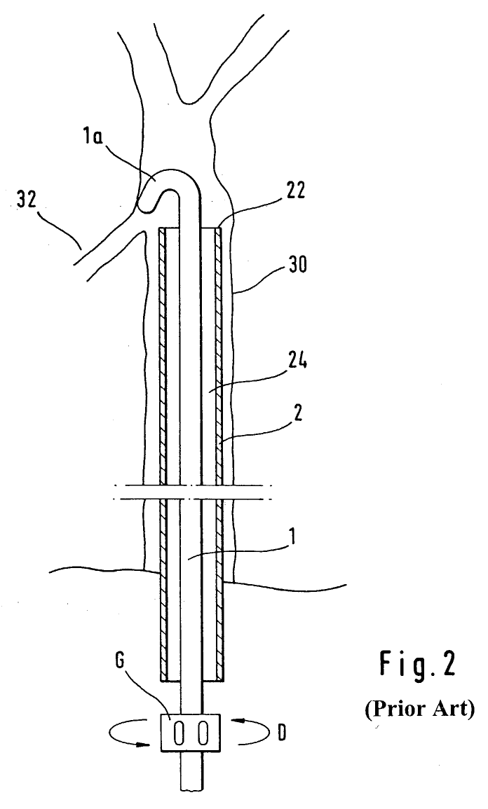

The device according to this invention for fixing and guiding a guide wire 1 and a catheter 2 is shown in FIG. 1a in the assembled state, and with the guide wire 1 firmly clamped in place, or fixed in its position. The proximal end of the guide wire, facing away from the patient, is marked 10, and the distal end of the guide wire is marked 12. The housing 7, the squeezer element 6, and the closure part 5 form the fixation device for holding, such as firmly clamping, the guide wire and for guiding the guide wire. The fixation device includes the housing 7 with the bore 74 that is continuous in the axial direction X and with the squeezer element 6 inserted regionally into the bore 74 of the housing 7. The squeezer element 6 likewise has an axially continuous bore 64. The squeezer element 6 is fixed in the housing 7 by the closure part 5, which is secured releasably to the housing 7 when an axial pressure is exerted onto the squeezer element 6. The closure part 5 also has a continuous axial bore 54, which receives the guide wire 1. Adjoining the distal end of the closure part 5 is a catheter adapter 4 with a formed-on catheter connector 41, which has an axially continuous bore 44 for the passage through it of the guide wire 1. The catheter adapter 4 is releasably secured to the closure part 5. The catheter 2 is connected to the catheter connector 41 of the catheter adapter 4, which connector is embodied as a flexible-tube coupling 41. The catheter 2 has a lumen 24, which is continuous from its proximal end 23 to its distal end 22, but the catheter can also have a closed distal tip. The catheter 2 is secured by its proximal end 23 to the catheter connector 41 of the catheter adapter 4. The guide wire 1 is likewise introduced into the catheter 2 up to the distal end region thereof. The fixation device of this invention for the guide wire 1 and the catheter 2 of FIG. 1a is used to connect and secure the catheter 2 and simultaneous fix and secure the guide wire 1 to the fixation device, which is assembled by the housing 7, squeezer element 6, closure part 5, and catheter adapter 4. The housing 7 serves as a grip and makes manipulation of the device as a unit possible. The fixation device with the catheter adapter 4 makes the simultaneous change of position of the catheter 2 and guide wire 1 possible; the position of the guide wire 1 is defined beforehand in the catheter 2. It is possible to fix the distal end of the catheter 2 relative to the distal end of the guide wire 1 in advance by a rigid coupling of the guide wire 1 and catheter 2, with the fixation device. In a subsequent simultaneous and common motion of the catheter 2 and guide wire 1 in the direction of the arrow P1 by the device, axial shifting of the guide wire 1 relative to the catheter 2 is avoided.

Instead of the catheter adapter 4 shown in FIG. 1 a with the formed-on flexible-tube coupling, it is also possible, for example, to use a catheter adapter, as shown in FIG. 8, with a catheter connector which is embodied as a stepped adapter with a stepped connector 42, or a catheter adapter with a catheter connector as in FIG. 9, which is embodied as a double adapter with a Luer-lock connector.

It is also possible for the closure part 5, on its distal end region, to be embodied integrally with a catheter connector, as shown in FIG. 1b. No separate catheter adapter 4 as in FIG. 1a is provided. Instead, the distal end 50a of the closure part 5 is embodied directly as a connector for the catheter 2, embodied for example as a flexible-tube coupling 41 or as a stepped connector or as a male or female Luer-lock connector.

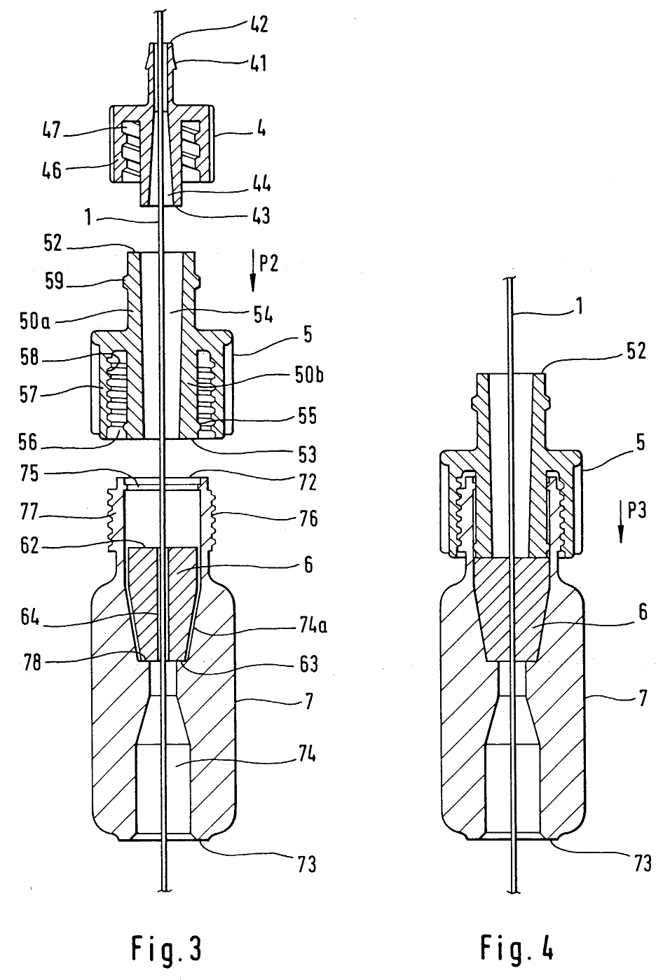

In FIG. 3, the parts provided according to this invention for the fixation device for holding and guiding both the guide wire 1 and the catheter 2 are shown in an exploded view and will be explained in further detail below in conjunction with this view. In it, the guide wire 1 is not yet fixed but it is in the opened position.

The housing 7 has a bore 74, which is continuous from the proximal end 73 to the distal end 72 of the housing 7 and which beginning at the distal end 72 tapers in stages toward approximately the middle, where on the shoulder formed it forms a bearing face 78 for the squeezer element 6. The collar 77, embodied in the distal end region 72 of the housing 7, has a smaller outer diameter than the remainder of the body of the housing 7, and on the outside can have a thread 76 so that it can be screwed to the closure part 5. The housing 7 simultaneously serves as a grip for the fixation device and can, as shown for instance in FIG. 7, be embodied on the outside with a plurality of axially extending ribs 79, spaced apart uniformly from one another, which enable secure grasping and prevent slipping. The housing 7 that acts as a grip can, however, have other forms. For good manipulation, the diameter of the housing 7 or grip should amount to at least approximately 12 mm, and the thickened part of the housing 7 should have a length of at least 15 mm. The housing 7, the closure part 5, and the catheter adapter 4 are all preferably made from a thermoplastic as an injection-molded part.

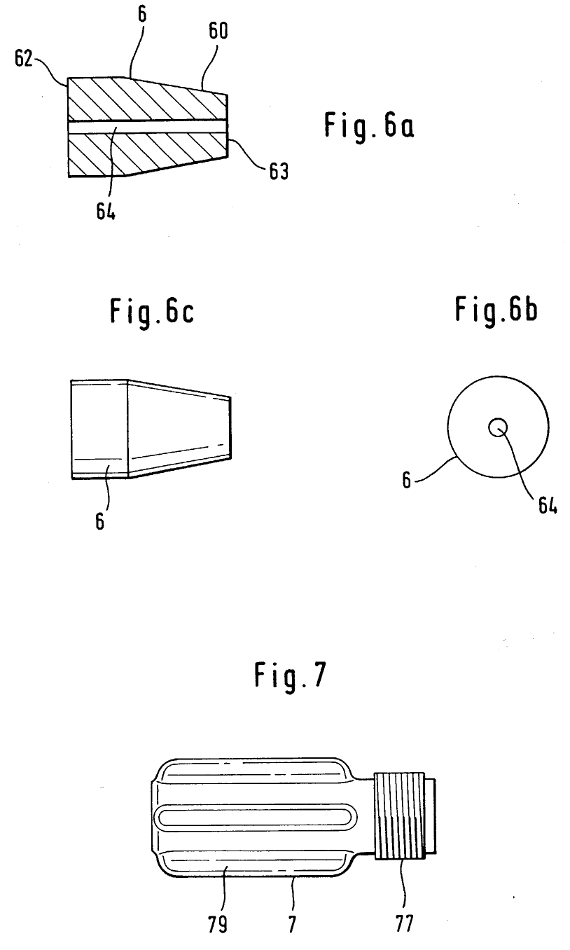

The bore 74 of the housing 7 tapers toward the bearing face 78 across the region 74a. In this region, the squeezer element 6, which in this example is elastic, is inserted into this region and has a suitable outer contour. The squeezer element 6 can, for example, be made from an elastic and/or thermoplastic plastic and is shown as an example in FIGS. 6a-6c in a longitudinal section, plan view and front view. The squeezer element 6 rests with its proximal end 63 on the bearing face 78 of the housing 7, and it ends with its distal end 62 still inside the bore 74 of the housing 7. The squeezer element 6 has a central axially continuous bore 64 from the distal end to the proximal end for the passage through it of the guide wire 1, as shown in FIG. 3. The closure part 5 can be a connecting piece part and punch for the squeezer element 6. When the closure part 5 is screwed onto the collar 77 of the housing 7 in the direction of the arrows P2, P3, the squeezer element 6 is axially crushed, as shown in FIG. 4. Thus the bore 64 of the squeezer element 6 closes and the guide wire 1 located in the bore 64 is firmly clamped in the squeezer element 6. The closure part 5 has a continuous nozzle stub 50a, with a continuous bore 54 from the distal end 52 to the proximal end 53. The distal end 52 of the nozzle stub 50a of the closure part 5 is embodied as a Luer-lock connecting piece and has beads 59, protruding on the outside, for interlocking with the catheter adapter 4. Toward the proximal end, the nozzle stub 50b also has on the outside a detent bead 55, and is also surrounded by the coaxially embodied jacket 57, forming the annular chamber 56. A female thread 58 is embodied on the inside of the jacket 57, so that the closure part 5 can be guided with the annular chamber 56 over the collar 77 of the housing 7 and screwed onto it, as shown in FIG. 4. Near the distal end 72 of the housing 7, on the inside of the collar 77, an annular bead 75 is embodied, by way of which the closure part 5 locks in detent fashion to the detent bead 55, so that while the rigid connection between the closure part S and the housing 7 that can be created by screwing is releasable, nevertheless the housing and closure part attach movably to one another via the annular bead 75 and the detent bead 55.

For connection of the catheter 2, the catheter adapter 4 with the catheter connector 41 is secured releasably to the distal end 52 of the closure part 5. The catheter adapter 4 has a continuous lumen 44 from the proximal end 43 to the distal end 42. The connecting piece is embodied toward the proximal end 43 as a Luer-lock connecting piece 46, 47 and toward the distal end as a flexible-tube coupling 41. The assembly direction is indicated by P2 in FIG. 3, and all the parts 4, 5, 6, 7 are threaded onto the guide wire 1, and only then is the squeezer element 6 inserted into the housing 7, after which the closure part 5 is screwed on and the squeezer element 6 is squeezed and the guide wire 1 is fixed. Then the catheter adapter 4 is secured to the closure part 5, unless previously done. The catheter 2 can now, as shown in FIG. 1, be secured by its proximal end 23 to the catheter connector 41 of the catheter adapter 4.

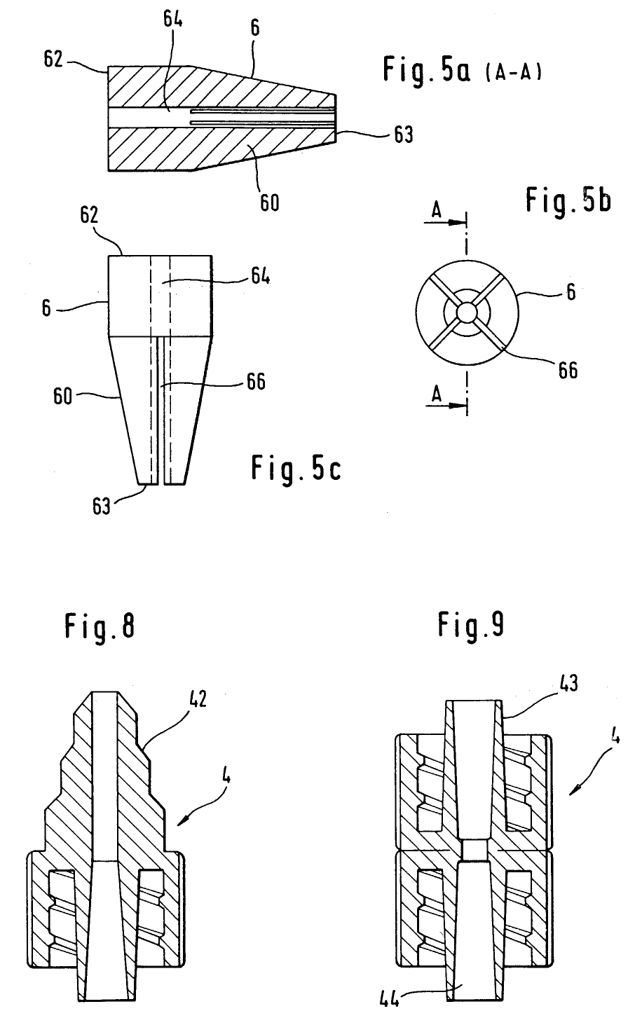

In FIGS. 5a-5c, still another possible embodiment is shown for a squeezer element 6, which is made from a hard material and is provided with additional slots 67 in the region of the catheter 60, which makes both compressibility and a contact-pressure force for the guide wire in the squeezer element 6 attainable.

German Patent Reference 202 10 509.1 and German Patent Reference 202 15 003.8, the priority documents corresponding to this invention, and their teachings are incorporated, by reference, into this specification.

What is claimed is:

1. In a device for holding and guiding a guide wire in a catheter, wherein the device is secured releasably to the guide wire, the device having a proximal end and a distal end, and having a recess continuous from the proximal end to the distal end of the device, the guide wire passable through the recess to introduce the catheter into a vessel or a body cavity of a patient, the improvement comprising: the catheter being releasably secured so that the device performs a common motion of the guide wire and the catheter in a previously defined position of the guide wire relative to the catheter.

2. In the device of claim 1, further comprising a fixation device for passing through and fixing the guide wire, the fixation device including a housing, a squeezer element that is insertable into the housing, and a closure part releasably connected to the housing, and a releasable securement of the catheter disposed on the distal end of the fixation device.

3. In the device of claim 2, further comprising a catheter adapter for securing the catheter, the catheter adapter releasably secured to the distal end of the fixation device and having a catheter connector for connection of the catheter.

4. In the device of claim 2, wherein the fixation device for securing the catheter is on the distal end integrated with a catheter connector for a connection of the catheter.

5. In the device of claim 4, wherein the catheter connector is a flexible-tube coupling.

6. In the device of claim 4, wherein the catheter connector is a Luer-lock connector.

7. In the device of claim 4, wherein the catheter connector is a stepped connector.

8. In the device of claim 7, wherein the fixation device for the guide wire includes a housing with a grip and a continuous bore for passing the guide wire, a squeezer element with a second continuous bore for passing the guide wire, and a closure part acting as a punch for action on the squeezer element, with a third continuous bore, and the squeezer element is insertable into the continuous bore of the housing, and the closure part is releasably securable to the housing by exerting a pressure on the squeezer element, and the bores of the housing and of the squeezer element and of the closure part extend in a common axis.

9. In the device of claim 8, wherein the distal end of the closure part has a connector for the connection of one of the catheter adapter and the catheter.

10. In the device of claim 9, wherein the closure part is releasably connectable on the proximal end to the housing with at least one of a screw connection, a bayonet mount, a detent connection and a clamping connection.

11. In the device of claim 10, wherein the closure part has a tubular stub that is continuous from a stub proximal end to the distal end and has a continuous bore, and a proximal end region of the tubular stub is insertable into the bore of the housing, and an end face on the stub proximal end of the tubular stub acts as a contact-pressure face upon the squeezer element, and the stub proximal end of the tubular stub is surrounded with a clearance by a coaxially disposed jacket that has on an inside one of a thread and a profiling for releasable connection to the housing.

12. In the device of claim 11, wherein the housing is one of cylindrically and spherically shaped and has on an outside surface one of axially extending, protruding ribs about a circumference and fluting.

13. In the device of claim 12, wherein the distal end region of the housing has an offset collar with a smaller diameter than a remainder of the housing, and on an outside the collar has profiling for positively engaging the closure part.

14. In the device of claim 13, wherein the collar can be introduced into the annular chamber formed on the closure part between the jacket and the proximal nozzle stub.

15. In the device of claim 14, wherein the continuous bore of the housing is tapered in a stepped fashion beginning at least at the distal end of the housing, toward a middle portion of the housing, and a bearing face is formed on a step shoulder, for the squeezer element.

16. In the device of claim 15, wherein the squeezer element is of an elastic material.

17. In the device of claim 15, wherein the squeezer element is of a hard material that is resilient and compressible.

18. In the device of claim 17, wherein one of a catheter adapter with a catheter connector is releasably secured to the distal end of the fixation device and a catheter connector is formed onto the distal end of the fixation device and the catheter is connected to the catheter connector, a position of the catheter relative to the fixation device is defined, and the guide wire guided through the fixation device is squeezed in the squeezer element by securing the closure part to the housing and is fixed in position relative to the fixation device and to the secured catheter, and the common motion change of position of the catheter and the guide wire is performed while maintaining a previously defined position of the guide wire relative to the catheter.

19. In the device of claim 1, further comprising a catheter adapter for securing the catheter, the catheter adapter releasably secured to the distal end of a fixation device and having a catheter connector for connection of the catheter.

20. In the device of claim 1, wherein a fixation device for securing the catheter is on the distal end integrated with a catheter connector for a connection of the catheter.

21. In the device of claim 1, wherein a catheter connector for a connection of the catheter is a flexible-tube coupling.

22. In the device of claim 1, wherein a catheter connector for a connection of the catheter is a Luer-lock connector.

23. In the device of claim 1, wherein a catheter connector for a connection of the catheter is a stepped connector.

24. In the device of claim 1, wherein a fixation device for the guide wire includes a housing with a grip and a continuous bore for passing the guide wire, a squeezer element with a second continuous bore for passing the guide wire, and a closure part acting as a punch for action on the squeezer element, with a third continuous bore, and the squeezer element is insertable into the continuous bore of the housing, and the closure part is releasably securable to the housing by exerting a pressure on the squeezer element, and the bores of the housing and of the squeezer element and of the closure part extend in a common axis.

25. In the device of claim 24, wherein the distal end of the closure part has a connector for the connection of one of the catheter adapter and the catheter.

26. In the device of claim 7, wherein the closure part is releasably connectable on the proximal end to the housing with at least one of a screw connection, a bayonet mount, a detent connection and a clamping connection.

27. In the device of claim 7, wherein the closure part has a tubular stub that is continuous from a stub proximal end to the distal end and has a continuous bore, and a proximal end region of the tubular stub is insertable into the bore of the housing, and an end face on the stub proximal end of the tubular stub acts as a contact-pressure face upon the squeezer element, and the stub proximal end of the tubular stub is surrounded with a clearance by a coaxially disposed jacket that has on an inside one of a thread and a profiling for releasable connection to the housing.

28. In the device of claim 7, wherein the housing is one of cylindrically and spherically shaped and has on an outside surface one of axially extending, protruding ribs about a circumference and fluting.

29. In the device of claim 7, wherein the distal end region of the housing has an offset collar with a smaller diameter than a remainder of the housing, and on an outside the collar has profiling for positively engaging the closure part.

30. In the device of claim 7, wherein the collar can be introduced into the annular chamber formed on the closure part between the jacket and the proximal nozzle stub.

31. In the device of claim 7, wherein the continuous bore of the housing is tapered in a stepped fashion beginning at least at the distal end of the housing, toward a middle portion of the housing, and a bearing face is formed on a step shoulder, for the squeezer element.

32. In the device of claim 7, wherein the squeezer element is of an elastic material.

33. In the device of claim 7, wherein the squeezer element is of a hard material that is resilient and compressible.

34. In the device of claim 1, wherein one of a catheter adapter with a catheter connector is releasably secured to the distal end of the fixation device and a catheter connector is formed onto the distal end of the fixation device and the catheter is connected to the catheter connector, a position of the catheter relative to the fixation device is defined, and the guide wire guided through the fixation device is squeezed in the squeezer element by securing the closure part to the housing and is fixed in position relative to the fixation device and to the secured catheter, and the common motion change of position of the catheter and the guide wire is performed while maintaining a previously defined position of the guide wire relative to the catheter.
